(19) United States
(12) Patent Application Publication
(10) Pub. No.: US 2026/0232874 A1
(43) Pub. Date: Aug. 13, 2026

(54) A METHOD FOR PROVIDING AN IMPROVED HEPARIN FUNCTIONALIZED SURFACE

(71) Applicant: CORLINE BIOMEDICAL AB, Uppsala (SE)

(72) Inventors: Fredrik CARLSSON, Uppsala (SE); Ulrika JOHANSSON, Uppsala (SE); Ingrid JÖNSSON, Uppsala (SE); Josefin SJÖGREN, Solna (SE)

(21) Appl. No.: 19/160,023

(22) PCT Filed: Mar. 28, 2024
(86) PCT No.: PCT/SE2024/050290

§ 371 Date: Aug. 26, 2025

(30) Foreign Application Priority Data
Mar. 30, 2023 (SE) 2350373-3

(51) Int. Cl. A61L 31/10; A61L 31/04; A61L 31/16

(52) U.S. Cl. CPC A61L 31/10; A61L 31/048; A61L 31/16; A61L 2300/232; A61L 2300/608; A61L 2420/02; A61L 2420/08

(57) ABSTRACT

The present invention relates to a method of preparing a multi-layered heparin conjugate coating on a substrate where the method facilitates better adhesion to the substrate surface and increase in the number of available heparin molecules on said surface.

### TECHNICAL FIELD OF THE INVENTION

The present invention relates to the field of methods for coating devices, more particularly medical devices, with a heparin-functionalized surface. The invention also relates to devices comprising such surfaces, wherein such devices have been produced by a method according to the invention. The present invention furthermore relates to uses and methods of such coated devices within the field of medical implants.

### BACKGROUND OF THE INVENTION

The present invention concerns a method for providing an improved heparin-functionalized coating on a surface, such as a synthetic surface or a biological scaffold surface.

Heparin has a long record as a clinically accepted anticoagulant by acting as a potent accelerator of antithrombin, a naturally occurring protein in blood that is capable of inhibiting a number of coagulation enzymes including thrombin. Hirudin is an example of a direct inhibitor of thrombin. The use of heparin or hirudin is associated with a less than negligible risk of bleeding.

A macromolecular conjugate composed of multiple heparin chains covalently linked to an inert main chain has been utilized for modifying both artificial (WO93/05793) and biological surfaces (WO00/45837) in such a way that these surfaces present permanently surface-bound heparin so as to mimic the chemical constitution of the blood vessel endothelium, which carries surface-localised heparan sulphate.

WO2013095270 concerns a heparin conjugate solution for treating blood vessels. The document further relates to the use of the heparin conjugate as a medicament and a method of coating tissue using the conjugate.

There is still a need in the art to provide improved methods for coating surfaces of medical devices with biocompatible heparin-containing coatings. Such coatings may assist in the healing process of such surfaces, or simply assist in avoiding a reaction from the body if the surface introduced into the human body is a non-biological surface.

### SUMMARY OF THE INVENTION

The above problems have now been overcome or at least mitigated by the provision herein of a method of providing a heparin-functionalized coating on a surface.

Accordingly, in a first aspect the present invention relates to a method of providing a heparin-functionalized coating on a surface, wherein said method comprises the steps of:
i) providing a surface having a coating comprising one or more layers wherein each layer comprises a polyallylamine (PAA) primer layer and a heparin conjugate layer,
ii) incubating said coating in a second borate buffer solution,
iii) incubating the coating of step ii) with an acylating solution comprising an acylating agent,
wherein said second borate buffer solution has a pH higher than 7;
wherein said acylating solution comprises an acylating agent at a concentration of at least 0.2 vol %, and
wherein the incubation of said coating in the second borate buffer solution in step ii) is performed for at least 10 minutes.

In a second aspect, there is also provided herein a heparin-functionalised surface comprising a coating obtained by a method as disclosed herein.

In a third aspect, there is also provided a device, such as a stent, catheter, vascular graft or a tubing, comprising a heparin-functionalized surface obtained as disclosed herein.

In a fourth aspect, there is provided a heparin-functionalised surface as disclosed herein, wherein said coating present on said surface is for use in preventing blood coagulation and/or for promoting anticoagulant properties and/or anti-complement activating properties in a subject in need thereof.

In a fifth aspect, there is provided the use of a device as disclosed herein as a medical implant.

In a sixth aspect, there is provided a method for preventing blood coagulation and/or for promoting anticoagulant properties and/or anti-complement activating properties in a subject in need thereof, said method comprising the steps of: i) providing a device comprising a heparin-functionalized surface as disclosed herein, and ii) implanting said device into the subject in need thereof.

In a seventh aspect, there is provided a method of providing an improved heparin-functionalized coating on a surface wherein the coating comprises one or more layers, wherein each layer comprises a primer layer and a heparin conjugate layer, wherein the method comprises the steps of: i) providing a surface, ii) optionally cleaning the surface, optionally by treating said surface with ammonium persulphate and rinsing said surface with an aqueous solution or by treating the surface with an alcohol in an ultrasound bath, iii) priming said surface by incubating said surface in a first borate buffer solution comprising a polyallylamine (PAA) primer, wherein the first borate buffer solution leaves a primer layer, iv) optionally rinsing the primer layer, v) incubating the primer layer obtained in the previous step in a heparin conjugate solution forming a heparin conjugate layer, vi) optionally rinsing the surface of step v), vii) optionally incubating the heparin conjugate layer obtained in the previous step in the first borate buffer solution comprising a PAA primer leaving an additional primer layer, viii) optionally rinsing the surface of step vii), ix) optionally incubating said additional primer layer in the heparin conjugate solution forming an additional heparin conjugate layer, x) optionally rinsing the surface of step ix); xi) optionally repeating steps vii) to x) in order to create an optional third or more layer(s) forming a coating, xii) incubating said coating in a second borate buffer solution, wherein said second borate buffer solution has a pH higher than 7, preferably 8 to 12, preferably 9 to 11.5, more preferably 10-11, more preferably about 10.5, and xiii) incubating the coating of step xii) by treating said surface with an acylating solution comprising an acylating agent, wherein said first borate buffer solution has a pH higher than 7, preferably 8 to 12, more preferably 8.5 to 11.0, more preferably 8.5 to 10, more preferably 8.5-9.5, more preferably about 9, wherein said acylating solution comprises an acylating agent at a concentration of at least 0.2 vol %, and wherein the incubation of said coating in the second borate buffer solution in step viii) is performed for at least 10 minutes.

### BRIEF SUMMARY OF THE FIGURES

FIG. 1 shows the layered system of a coating according to the present disclosure.

FIG. 2 illustrates method steps of a method according to the present disclosure

FIG. 3 shows the sampling scheme for the Antithrombin (AT) and Thrombin (TB) analysis of Example 1.

FIG. 4 shows available heparin vs. block position. Mean for all positions is illustrated as a straight black line. (part 1)

FIG. 5 shows AT concentration vs. block position. Mean for all positions is illustrated as a straight black line. (part 1)

FIG. 6 a) and b): a) Available heparin vs. block position with 5 and 30 minutes of incubation of borate buffer 10.5 and 0.3 vol % acetic anhydride. b) AT concentration vs. block position with 5 and 30 minutes of incubation of borate buffer 10.5 and 0.3 vol % acetic anhydride. Two different runs are presented. The mean for all the position in the first run is illustrated as straight line for 5 minutes and dotted line for 30 minutes. (part 1 and 2).

FIG. 7 shows available heparin for PE-pegs coated in block. Mean values, represented as straight lines, for the PE samples with a 0.3 vol % concentration of acetic anhydride and 5 and 30 minutes of incubation with the second borate buffer solution. Various lots of PE-samples coated in one block using a previous protocol (data not shown), using 0.1 vol % of acetic anhydride and 5 minutes incubation with borate buffer.

### DETAILED DESCRIPTION OF THE INVENTION

### Definitions

Herein the term “sulphated glycosaminoglycans” or simply referred to as “glycosaminoglycans” refers not only to the substances, which are normally included in the term, such as e.g. heparin, heparan sulphate, dermatan sulphate and chondroitin sulphate, but also fragments and derivatives of these substances which are functional for the purpose.

A “macromolecular conjugate of heparin” or a “heparin conjugate” as referred to interchangeably herein comprises multiple non-fractionated glycosaminoglycan (GAG), preferably heparin, molecules preferably covalently linked, preferably via single-point attachment, to an inert main chain.

The number of GAG molecules per conjugate should be at least ten but preferably between 20 and 100, that is 20 or more, or 50 or more or, or 70 or more, or 100 or less, or 80 or less.

A certain fraction of the GAG molecules will be engaged in binding to the surface while the remaining GAG molecules, not engaged in binding, are free to exert the biological activity of GAG. Due to multiple binding between the GAG molecules and the surface having affinity for GAGs, the binding strength of the macromolecular conjugate of GAGs will exceed that of ordinary GAG resulting in superior performance.

In order to obtain the advantageous combination of strong binding and retained biological activity it is preferable that multiple GAG chains are oriented in such a way that they are free to interact with the surface that has an affinity for GAGs and especially heparin. The GAG should be attached to the main chain preferably by single-point attachment.

One preferred macromolecular conjugate of GAG is the Corline Heparin Conjugate (CHC), which is composed of approximately 70 heparin molecules linked to the inert main chain (Obtainable from Corline Biomedical AB, Uppsala, Sweden). Accordingly, the number of heparin molecules may be between 40 and 75 per chain. A preferred conjugate is described in U.S. Pat. No. 5,529,986 which is hereby incorporated by reference. The macromolecular conjugate is an at least substantially water-soluble, biologically active conjugate (macromolecule), preferably in substantially pure form, comprising a substantially straight-chained organic homo- or heteropolymer having a number of functional groups distributed along the polymer backbone chain, via which groups of at least about 10 molecules from the group of sulphated glycosaminoglycans (GAG) in a non-active part thereof are anchored through covalent bonds. Such a conjugate may conceptually be described as a synthetic proteoglycan, the relative composition of which may be varied in a controllable way and adapted to the intended application.

The substantially linear polymer chain which is to function as the main chain for the glycosaminoglycan residues should, of course, be substantially biologically inert after the coupling of the glycosaminoglycan or -glycans in question, in the sense that it should be devoid of at least interfering biological activity. As is readily understood, in order to permit coupling of a plurality of glycosaminoglycan residues the main chain should be provided with a number of functional groups, such as e.g. amino, amide, sulphate, vinyl, carbonyl, nitro, thiol, hydroxyl or carboxyl groups, distributed along the chain and capable of, after optional modification, coupling of the glycosaminoglycan, either directly or via a coupling sequence. It is in this context to be noted that the GAG in question, depending on the method of producing the conjugate, may still have the terminal residue of its natural conjugate protein associated thereto, and that the binding then, of course, advantageously will take place via e.g. an amino acid in such a residue.

Further, the main chain, preferably a polymer chain, should preferably have a good solubility in water. At least it should, in accordance with what has previously been said about the conjugate, be at least substantially water-soluble after the coupling of the glycosaminoglycan groups. Specific polymer chains, which may be suitable for the purposes of the invention will readily be apparent to the skilled person after having taken part of the general inventive concept. This is, of course, also the case for the degree of branching on the polymer chain that may be permitted within the scope of the expression “substantially linear”.

Preferably the polymer chain is a natural or synthetic polypeptide, polysaccharide or an aliphatic polymer. The main chain may be an inert aliphatic compound. As specific non-limiting examples the main chains could be polylysine, poly ornithine, chitosan, polyimine and polyallylamine, starch, cellulose, chitin, hyaluronan, polyesters, polyethers, polyamides, polyamines, polyurethanes or combinations or mixtures thereof.

With regard to the fact that it is usually desired that the glycosaminoglycan will maintain its biological activity after the binding to the polymer main chain, it is preferred that each glycosaminoglycan molecule is bound terminally and by only a single bond to the main polymer. For example, the glycosaminoglycan may be bound to the main chain via an amino acid, and then preferably a terminal amino acid, but also free amino groups of a glucosamine unit may be used. The latter may exist free as such or may have been liberated through desulphation or deacetylation. Particularly in the case that an amino-functional polymer is used as the main chain, it may in some cases, especially when the main chain is sparsely substituted with glycosaminoglycans, be favourable to block the remaining free amino groups, which, for example, may be executed by acetylation. An alternative approach might be to substitute a desired number of amino groups with e.g. methyl groups before attaching the glycosaminoglycans.

The macromolecular heparin conjugate of the invention preferably has a molecular weight higher than 70 kDa. Preferably, the molecular weight of the macromolecular heparin conjugate is higher than 200 kDa, more preferably higher than 400 kDa, and even more preferably higher than 600 kDa. Other molecular weights are also envisaged herein.

The conjugate, when added to the surface disclosed herein, may be dissolved in an aqueous solution in a concentration range of 0.001-10 mg/ml, such as 0.001 mg/ml or more, or 0.01 mg/ml or more, or 0.1 mg/ml or more, or 1 mg/ml or more, or 3 mg/ml or more, or 10 mg/ml or less, or 7 mg/ml or less, or 5 mg/ml or less. The aqueous solution could be a buffer solution and the buffer could be any physiologic buffer for example a phosphate buffer, PBS.

The material of the surface may be any suitable material and may be both biological (e.g. a biological scaffold material prepared from decellularized tissue) or non-biological (e.g. metal, polymeric or ceramic). The surface may be a surface of a metallic or polymeric material. The metallic surface may be a nitinol (nickel titanium alloy) or platinum iridium alloy, but is not limited thereto. The metallic surface may also comprise steel or gold, or whatever deemed suitable for the intended purpose. The polymeric material may be selected from polyethylene, polypropylene, polystyrene, polyvinyl chloride, polycarbonate, polyurethane, polymethacrylate, polytetrafluoroethylene, cellulose or carboxymetylcellulose.

In addition, the substrate material to be coated as described herein may in principle be any material that is desired to be made biocompatible, provided that its surface is or may be made cationic. As described previously, the invention may apply to a body-foreign material, such as various polymers, metals and ceramics. It may also apply to tissue-like materials such as scaffolds prepared from decellularized extracellular matrix that may offer advantages to fully synthetic materials.

Various methods—for making a substrate surface cationic are well known. Treatment with polyimine has proved to be a suitable method, but also other polyamines, such as e.g. polylysine, chitosan or polyallylamine, may be used, as will be described in the working examples below.

Types of medical devices which may be coated using the present invention are tubings, stents, catheters, vascular grafts, membranes and filters, but are not limited thereto. Accordingly, a device, or a medical device, as disclosed herein may be used as a medical implant, sometimes referred to only as an implant. These terms are sometimes also used interchangeably herein, depending on the purpose. Some implants may be prosthetic, i.e., intended to replace missing body parts, others may provide support to body organs or tissues, deliver medicaments or provide other body monitoring functions. Implants may be placed permanently in the body, or they may be removed after some time when they are no longer needed.

Other examples of devices that may be coated using the present invention are ventricular assist devices, extracorporeal membrane oxygenators, stents, mechanical heart valves or arteriovenous (AV) fistulas. Such coated devices may be blood-contacting and/or blood compatible meaning that they possess characteristics that make them suitable for contact with blood, e.g. they may prevent blood clotting by presenting anticoagulant properties. Herein, a heparin-functionalised surface produced by a method of the present disclosure possess such properties. A surface that is envisaged as being coated by a method herein is collagen. For example, three dimensional matrices of collagen or gelatine (hydrolysed collagen) are hydrated by immersion in a phosphate buffer solution (PBS) for at least one hour followed by immersion in PBS supplemented with CHC (a heparin conjugate solution as presented herein) at a concentration ranging from 0.01-10 mg/ml for a period of 5 to 60 minutes and then careful rinsing three times with PBS.

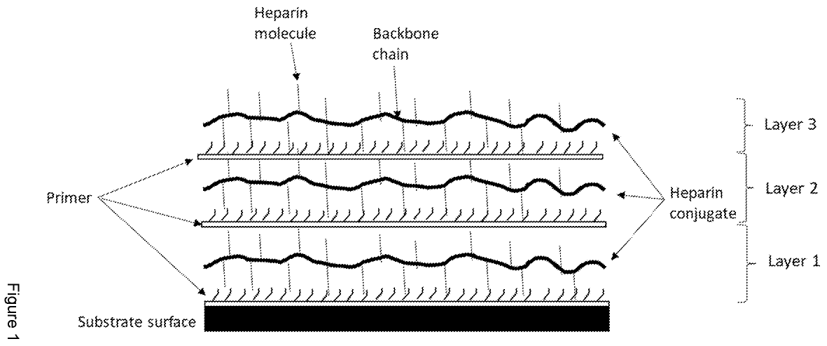

Referring to FIG. 1, the present invention relates to a method of preparing a coating on a surface where the method facilitates an improved heparin conjugate coating that is more resistant to wear. As viewed in FIG. 1, the coating is a multi-layered structure of at least two layers, preferably three layers, where each layer comprises a primer and heparin conjugate. The primer is used to attach the coating to the surface to coat and to separate each layer of heparin conjugates.

As seen in FIG. 1, the coating comprises two or more layers where each layer comprises a primer layer on top of which a heparin conjugate layer is arranged. In other words, on the surface of the material a primer layer is arranged and then a layer of heparin conjugate is arranged forming a first layer.

According to the present invention the primer is a polyallylamine having the general structure according to formula 1

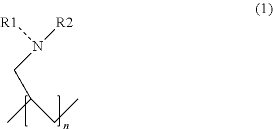

where R1 and R2 are individually selected from hydrogen or an alkyl group preferably a C1-C100 alkyl group. In one preferred embodiment R1 and R2 are hydrogen. In another preferred embodiment R1 is hydrogen and R2 is a C1-C100 alkyl group. In a preferred embodiment the polyallylamine has a molecular weight of 30,000-1,000,000 Da.

As the skilled person understands, even though the individual primer or heparin conjugate layers are arranged on top of or after each other the formed individual layers are at least partly mixed with or integrated with the previous individual layer. The number of layers of primer and heparin conjugate is at least two but preferably three or more.

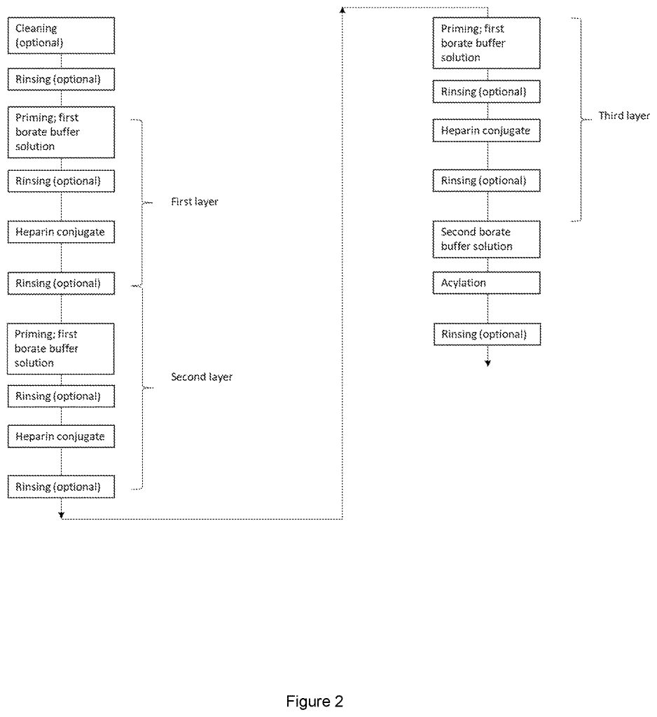

Referring now to FIG. 2, the method according to the present invention is a multistep method of coating a substrate surface with a heparin conjugate coating. A substrate surface is provided and preferably cleaned using any suitable method for said surface. When the substrate surface is a polymeric material such polyethylene or polypropylene the surface is preferably cleaned using ammonium persulphate and rinsed using an aqueous solution, or when the substrate surface is a metal by treating the surface with an alcohol in an ultrasound bath or sonication bath. The cleaned surface is then rinsed with an aqueous solution preferably two or more times.

A first borate buffer solution is then allowed to incubate the surface for a first period of time. The first borate buffer solution comprises the polyallylamine primer (PAA) as defined above. Herein, the concentration of the polyallylamine primer (PAA) of said first buffer solution may be about 0.15-0.35 mg/ml, preferably about 0.20-0.30 mg/ml, more preferably about 0.25 mg/ml. The first period of time is preferably at least 10 minutes, more preferably at least 15 minutes. Optionally the first primer layer is rinsed in order to remove any excess of primer. The rinsing is preferably done using an aqueous solution and is preferably repeated at least two times.

Thereafter, there is an incubation of the first primer layer in a heparin conjugate solution forming a first heparin conjugate layer. The heparin conjugate solution is preferably a buffer solution, preferably an acetate buffer solution having a pH lower than 5. The first primer layer may be incubated in the heparin conjugate solution for at least 5 minutes, preferably at least 10 minutes. Concentration of heparin conjugate in the heparin conjugate solution is preferably 0.01-0.1 mg/ml. The first heparin conjugate layer is then preferably rinsed with an aqueous solution and the rinsing is preferably repeated at least two times.

A second layer is prepared by first incubating the first heparin conjugate layer in the first borate buffer solution at a second priming temperature to create a second primer layer. Preferably the second primer layer is rinsed with an aqueous solution and the rinsing is preferably repeated at least two times. Then the second primer layer is incubated in the heparin conjugate solution forming a second heparin conjugate layer. The second primer may be incubated in the heparin conjugate solution for at least 5 minutes, preferably at least 10 minutes. The second heparin conjugate layer is then preferably rinsed with an aqueous solution and the rinsing is preferably repeated at least two times.

In order to create a third or additional layer the steps of incubating the layers in the first borate buffer solution, rinsing and incubating in the heparin conjugate solution is repeated until a wanted number of layers is obtained.

In order to further increase the number of available heparin groups on the surface of the coating the present inventors realized that acylating the coating is preferred. Prior to acylation the formed coating is incubated in a second borate buffer solution having a pH higher than 7, preferably 8 to 12, preferably 9 to 11.5, more preferably 10-11, more preferably about 10.5. The second borate buffer solution does not contain any primer. This step is believed to further cure the layered structure resulting in a more stable coating. Incubation in the second borate buffer solution is preferably done for at least 10 minutes, such as at least 15 minutes, preferably at least 20 minutes, more preferably at least 30 minutes such as about 35, 40, 45, 50, 55 minutes and up to about 60 minutes, alternatively wherein said incubation is allowed to proceed for multiple hours, such as overnight.

Thereafter the coating is treated with an acylating solution comprising an acylating agent, preferably an anhydride or acetyl halide, preferably acetic anhydride or acetyl chloride. The concentration of the acylating agent is at least 0.2 vol % and depends on the number of layers in the coating and on the substrate surface. The concentration may be at least 0.3 vol %, but preferably less than 50 vol %, preferably less than 20 vol %, preferably less than 10 vol %. Preferably, the concentration is about 0.2 vol % to 0.5 vol %, preferably 0.25-0.4 vol %.

As mentioned previously herein, one of the last steps of coating a device with a one or more layers of a primer and a heparin conjugate as disclosed herein, independent of the number of layers, is acylation of the remaining amines in the primer. By acylation the positively charged amines are exchanged to acyl groups that are not attracted to the negatively charged heparin chains on the heparin conjugate, resulting in freer non-covalently bonded heparin chains. The acylation step is preferably performed quickly to avoid hydrolysis of the acylation agent.

One aim of the present invention is to present an improvement of a method for coating a medical device with a heparin conjugate layer that if implemented will lead to an increase in the concentration of surface available heparin and antithrombin, thereby improving the coating process of medical devices.

Accordingly, it is disclosed herein a method of providing a heparin-functionalized coating on a surface, wherein the method comprises the steps of:
i) providing a surface having a coating comprising one or more layers wherein each layer comprises a polyallylamine (PAA) primer layer and a heparin conjugate layer,
ii) incubating said coating in a second borate buffer solution,
iii) incubating the coating of step ii) with an acylating solution comprising an acylating agent,
   wherein said second borate buffer solution has a pH higher than 7; wherein said acylating solution comprises an acylating agent at a concentration of at least 0.2 vol %, and wherein the incubation of said coating in the second borate buffer solution in step ii) is performed for at least 10 minutes. In one embodiment the pH of the second borate buffer solution is 8 to 12, preferably 9 to 11.5, more preferably 10-11, more preferably about 10.5.

The incubation of said coating in the second borate buffer solution in step ii) is performed for at least 10, such as at least 15 minutes, preferably at least 20 minutes, more preferably at least 30 minutes, such as about 35, 40, 45, 50, 55 minutes and up to about 60 minutes, alternatively wherein said incubation is allowed to proceed for multiple hours, such as overnight.

As mentioned elsewhere herein, said acylating solution comprises an acylating agent. The concentration of the acylating agent is at least 0.2 vol %, and depends on the number of layers in the coating and on the substrate surface. In one preferred embodiment the concentration is at least 0.3 vol %, but preferably less than 50 vol %, preferably less than 20 vol %, preferably less than 10 vol %. In one preferred embodiment the concentration is about 0.2 vol % to 0.5 vol %, preferably 0.25-0.4 vol %.

The acylating agent may be selected from acetic anhydride and acetyl chloride, preferably acetic anhydride.

Said first and second borate buffer solution as disclosed herein may comprise a compound selected from the group consisting of borate ester, a boric acid and/or a variant thereof, sodium tetraborate decahydrate, 1,3-dioxa-5-aza-2,4,6-triborinane and/or any combination thereof, but is not limited thereto. The first borate buffer is characterized by that it may be used for the build-up of each layer comprising a primer layer and a heparin conjugate layer, i.e., step i) of a method as disclosed herein comprising providing a surface having a coating comprising one or more layers wherein each layer comprises a polyallylamine (PAA) primer layer and a heparin conjugate layer.

The second borate buffer is characterized by that it may be used for the final stages of a method of the present disclosure, i.e., when the final layer comprising a primer layer and a heparin conjugate layer has been added to the surface comprising the one or more layers comprising a primer layer and a heparin conjugate layer.

As exemplified elsewhere herein, said heparin conjugate is preferably a substantially water-soluble biologically active conjugate comprising a main backbone chain of an inert aliphatic compound having a number of functional groups distributed along the backbone chain, via which functional groups at least about 20 and up to about 100, such as about 50 or 70 heparin molecules, in a non-active part of said molecule, are anchored through covalent bonds.

Herein, the step of providing a surface having a coating comprising one or more layers, wherein each layer comprises a polyallylamine (PAA) primer layer and a heparin conjugate layer, comprises the further steps of:
i) providing a surface,
ii) optionally cleaning the surface, optionally by treating said surface with ammonium persulphate and rinsing said surface with an aqueous solution or by treating the surface with an alcohol in an ultrasound bath,
iii) priming said surface by incubating said surface in a first borate buffer solution comprising a polyallylamine (PAA) primer, wherein the first borate buffer solution leaves a primer layer,
iv) incubating the primer obtained in the previous step in a heparin conjugate solution forming a heparin conjugate layer,
v) optionally incubating the heparin conjugate layer obtained in the previous step in the first borate buffer solution at a second priming temperature leaving an additional primer layer,
vi) optionally incubating said additional primer layer in the heparin conjugate solution forming an additional heparin conjugate layer,
vii) optionally repeating step v) to vi) order to create an optional third or more second or more layer(s);
   wherein said first borate buffer solution has a pH higher than 7, preferably 8 to 12, more preferably 8.5 to 11.0, more preferably 8.5 to 10, more preferably 8.5-9.5, more preferably about 9.

In said method, a rinsing step, optionally after step iii), iv), v) and/or vi), is performed, optionally with an aqueous solution.

As exemplified elsewhere herein, the incubating of said surface in a heparin conjugate solution may be performed for at least 15 minutes in each step. Preferably, the concentration of the polyallylamine (PAA) primer of said first borate buffer solution is about 0.15-0.35 mg/ml, preferably about 0.20-0.30 mg/ml, more preferably about 0.25 mg/ml.

As exemplified elsewhere herein, wherein the surface is a material, said material may be selected from the group consisting of a metal, such as an alloy of nickel and titanium, preferably nitinol, a plastic or polymeric material such as PVC (Polyvinyl chloride), PP (polypropylene), PC (polycarbonate), PU (polyurethane), PE (Polyethylene), PTFE (Polytetrafluoroethylene), PMMA (Poly(methyl methacrylate), a cellulosic material, and a ceramic material, and a biological scaffold material but is not limited thereto.

There is also provided herein a heparin-functionalised surface comprising a coating obtained by a method as disclosed herein.

There is also provided herein a device, such as a stent or a tubing, comprising a heparin-functionalized surface as disclosed herein. Said device may also be a vascular graft, a catheter, a membrane, a filter, a ventricular assist device, an extracorporeal membrane oxygenator, a mechanical heart valve or an arteriovenous (AV) fistula.

Said device may be a blood-contacting and/or a blood compatible device as described elsewhere herein.

There is also provided a heparin-functionalised surface as disclosed herein, wherein said coating is for use in preventing blood coagulation and/or for promoting anticoagulant properties and/or anti-complement activating properties in a subject in need thereof. Such a coating may also be described as thromboresistant. All in all, such properties may facilitate healing within the area implanted with a device of the present disclosure. The subject being implanted with a device of the present disclosure may suffer from cardiovascular disease or other diseases or disorders requiring assistance of an implant.

There is also provided herein a heparin-functionalised surface as disclosed herein, wherein said coating is for use in promoting tissue growth, healing and/or regeneration in a subject.

There is also provided the use of a device comprising a heparin-functionalized surface as disclosed herein as a medical implant.

There is also provided a method for preventing blood coagulation and/or for promoting anticoagulant properties and/or anti-complement activating properties in a subject in need thereof, said method comprising the steps of: i) providing a device comprising a heparin-functionalized surface as disclosed herein, and ii) implanting said device into the subject in need thereof.

More particularly, there is also provided a method of providing an improved heparin-functionalized coating on a surface wherein the coating comprises one or more layers, wherein each layer comprises a primer layer and a heparin conjugate layer, wherein the method comprises the steps of:
i) providing a surface,
ii) optionally cleaning the surface, optionally by treating said surface with ammonium persulphate and rinsing said surface with an aqueous solution or by treating the surface with an alcohol in an ultrasound bath,
iii) priming said surface by incubating said surface in a first borate buffer solution comprising a polyallylamine a (PAA) primer, wherein the first borate buffer solution leaves a primer layer.
iv) optionally rinsing the primer layer,
v) incubating the primer layer obtained in the previous step in a heparin conjugate solution forming a heparin conjugate layer,
vi) optionally rinsing the surface of step v)
vii) optionally incubating the heparin conjugate layer obtained in the previous step in the first borate buffer solution leaving an additional primer layer,
viii) optionally rinsing the surface of step viii)
ix) optionally incubating said additional primer layer in the heparin conjugate solution forming an additional heparin conjugate layer,
x) optionally rinsing the surface of step ix);
xi) optionally repeating steps vii) to x) in order to create an optional third or more layer(s) forming a coating,
xii) incubating said coating in a second borate buffer solution, wherein said second borate buffer solution has a pH higher than 7, preferably 8 to 12, preferably 9 to 11.5, more preferably 10-11, more preferably about 10.5, and
xiii) incubating the coating of step xii) by treating said surface with an acylating solution comprising an acylating agent,
   wherein said first borate buffer solution has a pH higher than 7, preferably 8 to 12, more preferably 8.5 to 11.0, more preferably 8.5 to 10, more preferably 8.5-9.5, more preferably about 9, wherein said acylating solution comprises an acylating agent at a concentration of at least 0.2 vol %, and wherein the incubation of said coating in the second borate buffer solution in step viii) is performed for at least 10 minutes.

The present disclosure will now be illustrated by the following experimental section, but it is not intended to be limited thereto.

### EXPERIMENTAL SECTION

### Materials and Methods

The below concerns a coating process optimization comprising increasing the concentration of acylating solution (acetic anhydride) and incubation time of the second borate buffer solution with a pH 10.5 during coating of medical devices with heparin conjugate-containing layers as disclosed herein.

### Example 1

Polyethylene (PE) samples were coated in block according to the below procedure.

The heparin conjugate used in the example is Corline heparin conjugate (CHC).

The following tests were performed:
1. Incubation time of second borate buffer with pH 10.5, 5 min+concentration of acetic anhydride (acylating solution) 0.3 vol %,
2. Incubation time of second borate buffer with pH 10.5, 30 min+concentration of acetic anhydride (acylating solution) 0.3 vol %,

All tests were conducted in a flow chamber where the solutions were recirculated for 15 minutes through or passed the test samples and all the samples were coated with three layers of primer and heparin conjugate (each layer comprising a primer and a heparin conjugate).

For each test a first borate buffer solution (pH 9.0-9.1) comprising an alkylated polyallylamine primer was prepared (0.25 mg primer/ml), a second borate buffer solution (pH 10.5-10.6) and a heparin conjugate solution (pH 4.0) comprising a heparin conjugate dissolved in an acetate buffer (0.051 mg heparin conjugate/ml) were prepared.

All the materials were first washed with ethanol in a sonication bath and rinsed with milli-q water prior to coating.

The following steps were taken:
1) Recirculating the first borate buffer solution through the samples to prime the sample surfaces.
2) Rinsing four times with milli-q water (no recirculation).
3) Recirculating the first heparin conjugate solution through the samples.
4) Rinsing two times with milli-q water (no recirculation).
5) Recirculating the first borate buffer solution through the samples.
6) Rinsing four times with milli-q water (no recirculation).
7) Recirculating the first heparin conjugate solution through the samples.
8) Rinsing two times with milli-q water (no recirculation).
9) Recirculating the first borate buffer solution through the samples.
10) Rinsing four times with milli-q water (no recirculation).
11) Recirculating the first heparin conjugate solution through the samples.
12) Rinsing two times with milli-q water (no recirculation).
13) Recirculating the second borate buffer solution through the samples for 5 minutes or 30 minutes at ambient temperature.
14) Prepare an acylating borate buffer solution (PH ~10.5, acetic anhydride conc. 0.3 vol % or 0.1 vol %, respectively) and recirculating the acylating buffer solution for 5 minutes or 30 minutes, respectively, through the samples.
15) Rinsing four times with milli-q water (no recirculation).
16) Drying the PE samples over night

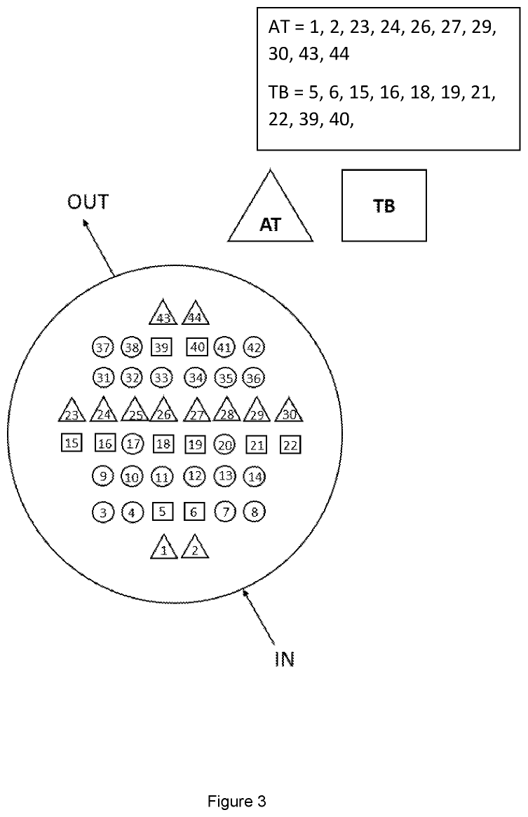

In FIG. 3 a sampling scheme for the Antithrombin (AT) and Toluidine blue (TB) analysis that was used is shown.

### Results

### Coating of PE Samples

### Part 1)

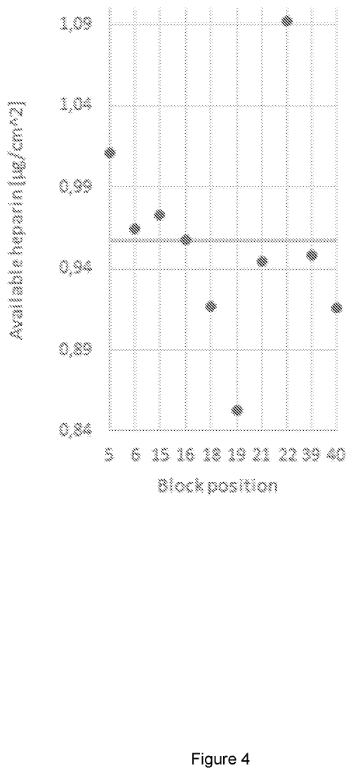

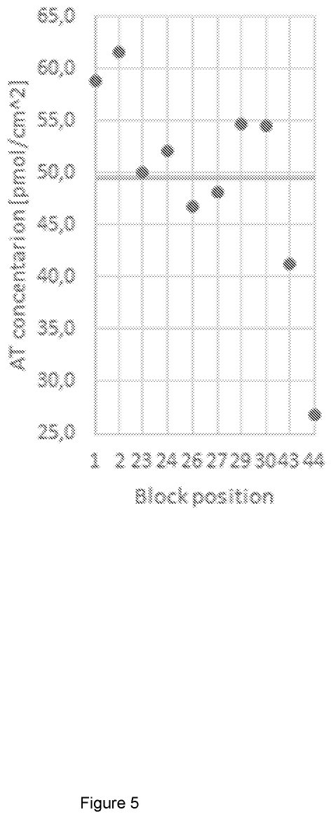

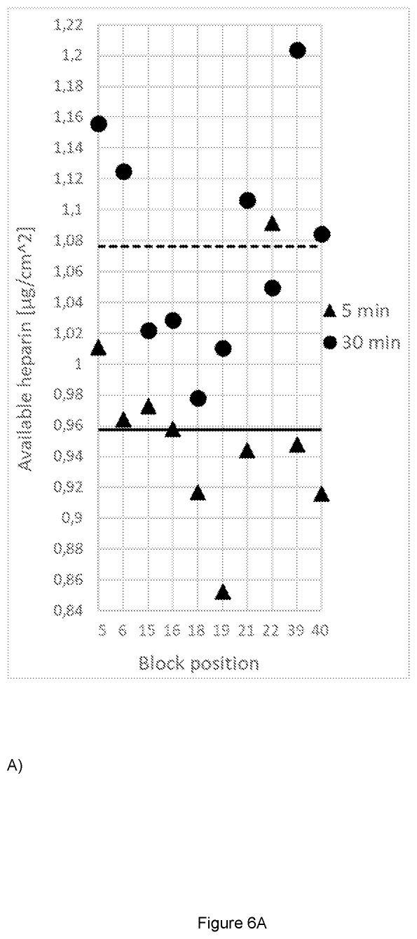

Using 0.3 vol % of the acetic anhydride, the available heparin and the AT concentration for the analysed PE-samples is presented in FIGS. 4 and 5 respectively, see FIG. 3 for block positions. The available heparin concentration is between 0.84-1.09 μg/cm².

In FIG. 4, comparing the results with regards to position, at the inlet positions 5 and 6 and outlet positions 39 and 40, available heparin concentration lies above and below the mean value but do not deviate as much from mean.

In FIG. 5, the available AT concentration range above 25 and below 65 μmol/cm². A clear difference is seen in the AT values where position 1 and 2 (near the inlet) and position 43 and 44 (near the outlet) deviates more from the mean value compared to the other positions.

### Part 2)

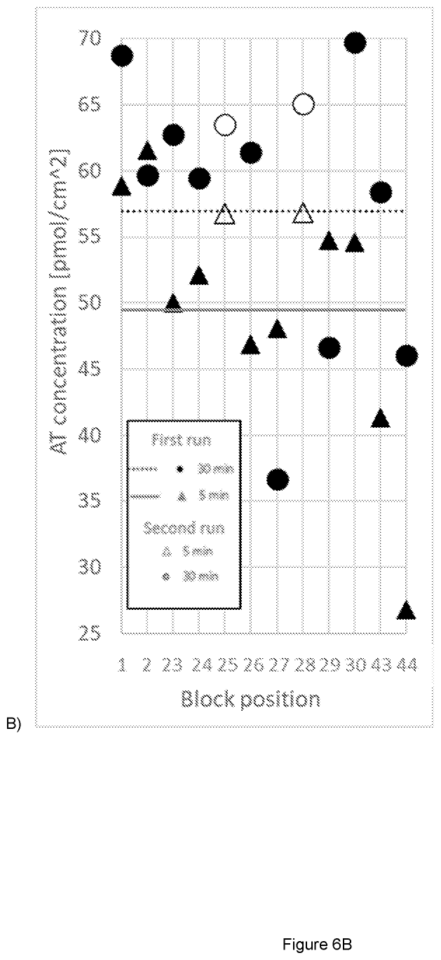

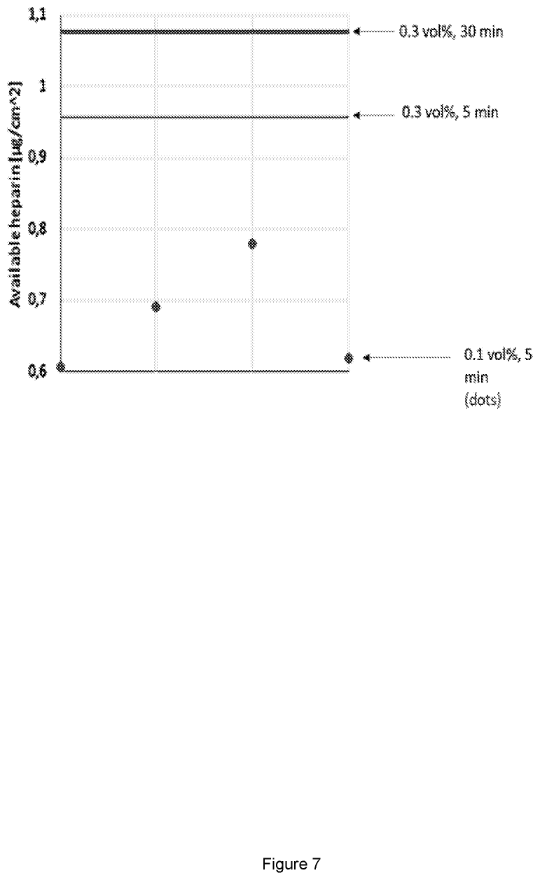

When 0.3 vol % of the acetic anhydride and an increased incubation time (30 min) of borate buffer pH 10.5 (second borate buffer) was used, see FIG. 6a-b. The results from PE-samples above was combined with results from an increased acetic anhydride 0.3 vol % and a prolonged incubation time from 5 to 30 minutes. This shows that this generates an increase in available heparin of 12% (FIG. 6a) and an increase in AT value between 13-15% (FIG. 6b) and the trend is that it also lowers the variability in results with regards to positions.

### Discussion

Illustrated with the PE-samples herein, both an increase of acetic anhydride concentration (from 0.1 vol % to 0.3 vol %) and pro-longed incubation time of second borate buffer pH 10.5 increased the heparin and AT concentration and seems also to keep the variation in the position in the blocks lower.

As an illustration of an optimized process with regards to available heparin, See FIG. 5. In FIG. 5 a comparison from old available data of PE-pegs coated in blocks with 0.1 vol % and 5 min incubation with second borate buffer with the PE-pegs coated with 0.3 vol % acetic anhydride and either 5 min incubation or pro-longed incubation of 30 min shows the real effect of an optimized CHS™ process. Here, the increased concentration of acetic anhydride and in combination with pro-longed incubation time with second borate buffer pH 10.5 results in an increase of available heparin of 42% and 59%, respectively.

1. A method of providing a heparin-functionalized coating on a surface, wherein the method comprises the steps of:
i) providing a surface having a coating comprising one or more layers wherein each layer comprises a polyallylamine (PAA) primer layer and a heparin conjugate layer,
ii) incubating said coating in a second borate buffer solution,
iii) incubating the coating of step ii) with an acylating solution comprising an acylating agent,
wherein the second borate buffer solution has a pH higher than 7;
wherein said acylating solution comprises an acylating agent at a concentration of at least 0.2 vol %, and
wherein the incubation of said coating in the second borate buffer solution in step ii) is performed for at least 10 minutes.

2. The method of claim 1, wherein the incubation of said coating in the second borate buffer solution in step ii) is performed for at least 15 minutes.

3. The method of claim 1, wherein said acylating solution comprises an acylating agent at a concentration of about 0.2 vol % to about 0.5 vol %.

4. The method of claim 1, wherein the acylating agent is selected from acetic anhydride and acetyl chloride.

5. The method of claim 1, wherein said heparin conjugate is a substantially water-soluble biologically active conjugate comprising a main backbone chain of an inert aliphatic compound having a number of functional groups distributed along the backbone chain, via which functional groups at least about 20 and up to about 100, in a non-active part of said molecule, are anchored through covalent bonds.

6. The method of claim 1, wherein the step of providing a surface having a coating comprising one or more layers, wherein each layer comprises a polyallylamine (PAA) primer layer and a heparin conjugate layer, comprises the steps of:
i) providing a surface,
ii) optionally cleaning the surface, optionally by treating said surface with ammonium persulphate and rinsing said surface with an aqueous solution or by treating the surface with an alcohol in an ultrasound bath,
iii) priming said surface by incubating said surface in a first borate buffer solution comprising a polyallylamine (PAA) primer, wherein the first borate buffer solution leaves a primer layer,
iv) incubating said primer layer of the previous step in a heparin conjugate solution forming a heparin conjugate layer,
v) optionally incubating the heparin conjugate layer obtained in the previous step in the first borate buffer solution at a second priming temperature leaving an additional primer layer,
vi) optionally incubating said additional primer layer in the heparin conjugate solution forming an additional heparin conjugate layer,
vii) optionally repeating step v) to vi) in order to create an optional third or more layer(s);
wherein said first borate buffer solution has a pH higher than 7.

7. The method of claim 6, wherein a rinsing step, optionally after step iii), iv), v) and/or vi), is performed.

8. (canceled)

9. The method of claim 1, wherein said first and second borate buffer solution comprises a compound selected from the group consisting of borate ester, a boric acid and/or a variant thereof, sodium tetraborate decahydrate, 1,3-dioxa-5-aza-2,4,6-triborinane and/or any combination thereof.

10. The method according to claim 6, wherein the concentration of the polyallylamine (PAA) primer of said first borate buffer solution is about 0.15-0.35 mg/ml.

11. The method of claim 1, wherein the surface is a material, wherein said material is selected from the group consisting of a metal, a plastic or polymeric material a ceramic material, and a biological scaffold material.

12. The method according to claim 1 wherein the second borate buffer solution has a pH of about 8 to 12.

13. A heparin-functionalised surface comprising a coating obtained by a method according to claim 1.

14. A device, such as a stent or a tubing, comprising a heparin-functionalized surface according to claim 13.

15. The device of claim 14, wherein said device is a blood-contacting and/or a blood compatible device.

16. The device of claim 14, wherein said device is a vascular graft, a catheter, a membrane, a filter, a stent, a ventricular assist device, an extracorporeal membrane oxygenator, a mechanical heart valve or an arteriovenous (AV) fistula.

17-18. (canceled)

19. A method for preventing blood coagulation and/or for promoting anticoagulant properties and/or anti-complement activating properties in a subject in need thereof, said method comprising the steps of:
i) providing a device comprising a heparin-functionalized surface of claim 14, and
ii) implanting said device into the subject in need thereof.

20. A method of providing an improved heparin-functionalized coating on a surface wherein the coating comprises one or more layers, wherein each layer comprises a primer layer and a heparin conjugate layer, wherein the method comprises the steps of:
i) providing a surface,
ii) optionally cleaning the surface, optionally by treating said surface with ammonium persulphate and rinsing said surface with an aqueous solution or by treating the surface with an alcohol in an ultrasound bath,
iii) priming said surface by incubating said surface in a first borate buffer solution comprising a polyallylamine (PAA) primer, wherein the first borate buffer solution leaves a primer layer.
iv) optionally rinsing the primer layer,
v) incubating the primer layer obtained in the previous step in a heparin conjugate solution forming a heparin conjugate layer,
vi) optionally rinsing the surface of step v)
vii) optionally incubating the heparin conjugate layer obtained in the previous step in the first borate buffer solution leaving an additional primer layer,
viii) optionally rinsing the surface of step vii),
ix) optionally incubating said additional primer layer in the heparin conjugate solution forming an additional heparin conjugate layer,
x) optionally rinsing the surface of step ix);
xi) optionally repeating steps vii) to x) in order to create an optional third or more layer(s) forming a coating,
xii) incubating said coating in a second borate buffer solution, wherein said second borate buffer solution has a pH higher than 7, and
xiii) incubating the coating of step xii) by treating said surface with an acylating solution comprising an acylating agent,
wherein said first borate buffer solution has a pH higher than 7,
wherein said acylating solution comprises an acylating agent at a concentration of at least 0.2 vol %, and
wherein the incubation of said coating in the second borate buffer solution in step viii) is performed for at least 10 minutes.

21. The method of claim 1, wherein the surface is a material, wherein said material is selected from the group consisting of a metal wherein the metal is an alloy of nickel and titanium.

22. The method of claim 1, wherein the surface is a material, wherein said material is a plastic or polymeric material wherein said plastic or polymer is selected from PVC (Polyvinyl chloride), PP (polypropylene), PC (polycarbonate), PU (polyurethane), PE (Polyethylene), PTFE (Polytetrafluoroethylene), PMMA (Poly(methyl methacrylate) and a cellulosic material.

23. The method according claim 1, wherein the second borate buffer solution has a pH of about 9 to 11.5.
